(19) United States
(12) Patent Application Publication
(10) Pub. No.: US 2026/0035321 A1
(43) Pub. Date: Feb. 5, 2026

(54) PROCESS FOR HYDROGENATING A HYROCARBON STREAM WITH ELECTROLYSIS

(71) Applicant: UOP LLC, Rosemont, IL (US)

(72) Inventors: James W. Harris, Palatine, IL (US); Ian G. Horn, Streamwood, IL (US); Ashish Mathur, Gurgaon (IN); Nirlipt Mahapatra, Gurgaon (IN); Travis Bowen, Crystal Lake, IL (US)

(21) Appl. No.: 19/262,849

(22) Filed: Jul. 8, 2025

Related U.S. Application Data
(60) Provisional application No. 63/678,031, filed on Jul. 31, 2024.

(51) Int. Cl. C07C 5/10; C25B 1/042; C25B 15/08

(52) U.S. Cl. CPC C07C 5/10; C25B 1/042; C25B 15/081; C07C 2601/16

(57) ABSTRACT

A process of hydrogenating an unsaturated hydrocarbon is disclosed. The process comprises passing a hydrocarbon feed stream comprising toluene to a hydrogenation reactor. A hydrogen stream is passed to the hydrogenation reactor. In the hydrogenation reactor, the hydrocarbon feed stream is hydrogenated in the presence of hydrogen and a hydrogenation catalyst to produce a hydrogenated effluent stream comprising methylcyclohexane. The hydrogenated effluent stream is indirectly contacted with a water stream to produce a steam stream. The steam stream is taken from the hydrogenation reactor. In an electrolyzer, hydrogen is separated from the steam stream to produce the hydrogen stream which is passed to the hydrogenation reactor.

### FIELD

The field is hydrogenating a hydrocarbon stream. The field may particularly relate to hydrogenating an unsaturated hydrocarbon stream.

### BACKGROUND

Hydrogen is expected to have significant growth potential because it is a clean-burning fuel. However, hydrogen production processes based on steam reforming, autothermal reforming, partial oxidation, or gasification of hydrocarbon or carbonaceous feedstocks are significant emitters of carbon dioxide. Government regulations and societal pressures are increasingly taxing or penalizing carbon dioxide emissions or incentivizing carbon dioxide capture. Consequently, there is significant interest in lowering the cost of hydrogen production using these processes and recovering the byproduct carbon dioxide for subsequent geological sequestration. Hydrogen from solar, wind, and water, which does not involve the production of carbon dioxide, could meet projected global energy demand in the future and play a vital role in reducing global warming. The recently renewed interest in alternative energy sources and energy carriers opens up new prospects for this process to be applied as a feed system for fuel cells, power generation and many more applications.

Hydrogen is a clean, energy efficient fuel for various mobile fuel-cell applications which has no adverse effects on the environment and human health. With increased global demand for hydrogen, solutions need to be developed to transport hydrogen especially to locations which are hydrogen depleted. Hydrogen generated by renewable energy sources is called green hydrogen. Green hydrogen is expected to be an important element in the future fuel economy and may need to be transported to locations as far as 8000 km from the source of generation.

There exists a huge regional disparity in the cost for production of hydrogen. A number of technologies have been developed for transporting hydrogen, including ammonia, liquid hydrogen, and liquid organic hydrogen carrier (LOHC) to address this disparity. Toluene-methylcyclohexane (MCH) is expected to be a significant player in LOHC considering its numerous advantages, such as easy integration into the existing fuel sector supply chain and distribution network, utilization in idle refinery assets, flexibility for co-processing, and higher relative safety handling.

LOHC involves the reversible dehydrogenation reaction of a hydrogen carrier such as methylcyclohexane (MCH) to produce toluene and hydrogen. It has been proposed as a solution for the storage, transportation, and distribution of hydrogen produced from renewable or non-renewable energy sources. For power generation, the hydrogen from this process is usually compressed for a downstream power generation unit. Usually, purity requirements for power generation unit is very stringent. Due to the relatively high cost associated with green hydrogen production, it is necessary to recover almost all of the hydrogen.

Hydrogen for LOHC can be produced by electrolytically splitting water using electrical and thermal energy. However, relative amounts of electrical and thermal energy vary with temperature. For high-temperature electrolysis such as by solid oxide, more thermal energy and less electrical energy is required. Some of the thermal energy for water electrolysis can be supplied by the hydrocarbon saturation process.

Accordingly, it would be desirable to have more effective and efficient ways to purify and transport hydrogen.

### BRIEF SUMMARY

A process of hydrogenating an unsaturated hydrocarbon is provided. The process comprises passing a hydrocarbon feed stream such as toluene to a hydrogenation reactor. A hydrogen stream is passed to the hydrogenation reactor. In the hydrogenation reactor, the unsaturated hydrocarbon is hydrogenated in the presence of hydrogen and a hydrogenation catalyst to produce a hydrogenated effluent stream such as methylcyclohexane. The hydrogenated effluent stream is indirectly contacted with one or more water streams to produce one or more steam streams. The steam streams are taken and passed to a hydrogen production unit comprising an electrolyzer. In the electrolyzer, hydrogen is produced from the steam stream, which is passed to the hydrogenation reactor.

### Definitions

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” or “directly” means that fluid flow from the upstream component enters the downstream component without passing through any other intervening vessel.

The term “indirect communication” means that fluid flow from the upstream component enters the downstream component after passing through an intervening vessel.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

The term “Cx” is to be understood to refer to molecules having the number of carbon atoms represented the subscript “x”. Similarly, the term “Cx-” refers to molecules that contain less than or equal to x and preferably x and less carbon atoms. The term “Cx+” refers to molecules with more than or equal to x and preferably x and more carbon atoms.

### BRIEF DESCRIPTION OF THE DRAWING

FIG. 1 is an illustration of an exemplary embodiment of a process of hydrogenating an unsaturated hydrocarbon.

FIG. 2 is an illustration of another exemplary embodiment of a process of hydrogenating an unsaturated hydrocarbon.

FIG. 3 is an illustration of yet another exemplary embodiment of a process of hydrogenating an unsaturated hydrocarbon.

### DETAILED DESCRIPTION

The present disclosure provides a process of hydrogenating an unsaturated hydrocarbon stream, which may comprise a toluene stream. The hydrogen used in the hydrogenation process may be produced from an electrolyzer. A desired approach for powering an electrolyzer is setting up a green energy production facility such as a solar farm or wind farm and feeding the power generated to the electrolyzer. The present process integrates hydrogenation with the electrolyzer using a hydrogenated effluent stream from a reactor to provide the thermal energy required in the electrolyzer for producing hydrogen. The present integrated process offsets some of the power needed for the electrolyzer for producing hydrogen. By offsetting the load of the electrolyzer, additional power can be routed to other uses or the power requirements can be reduced.

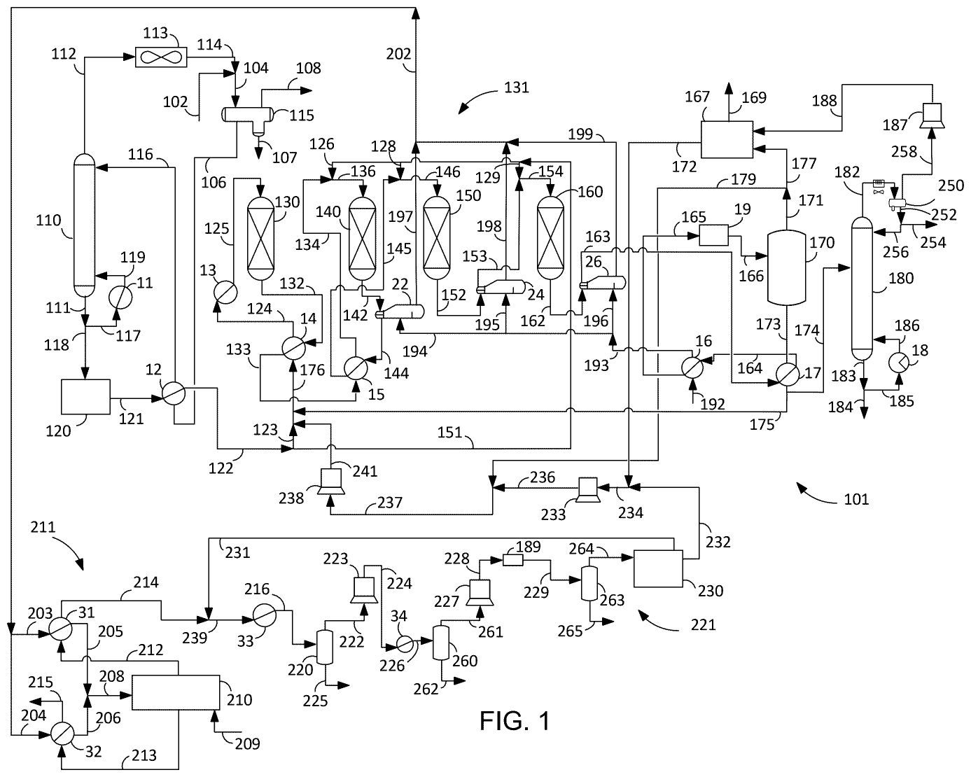

The FIG. 1 illustrates an embodiment of the process of hydrogenating an unsaturated hydrocarbon such as toluene. The process 101 comprises a hydrogenation section 131 comprising hydrogenation reactor 130, 140, 150 and 160, a hydrogen production section 211, and a purification section 221. As shown, an unsaturated hydrocarbon stream comprising an unsaturated hydrocarbon feed stream in line 102 is passed to the hydrogenation section 131. The hydrocarbon feed stream in line 102 may be provided from an external source such as a storage tank (not shown). If the hydrocarbon feed stream is imported from an external source, such as through a pipeline, land-going vehicle, or water-going vehicle, the feed stream may be exposed to oxygen and require treatment to remove the oxygen and/or oxygenated hydrocarbons, prior to introduction into the hydrogenation section 131. Such oxygen removal treatments may include but is not limited to oxygen stripping, heat soaking, caustic treatment, adsorption using activated alumina and/or molecular sieves, resins, fractionation, clay treatment, or any combination thereof. The hydrocarbon feed stream in line 102 may be a wet hydrocarbon feed stream. The wet hydrocarbon feed stream can be dried with a treatment step such as passing the wet hydrocarbon feed stream to a stripping column such as the oxygen stripping column 110. Other treatment steps for drying the wet hydrocarbon feed stream may include drying the wet hydrocarbon feed stream in a drier with a molecular sieve.

In an exemplary embodiment, the hydrocarbon feed stream in line 102 may be passed to an oxygen stripping column 110 to remove oxygen, oxygenated hydrocarbons and water from the feed stream. In an aspect, the hydrocarbon feed stream in line 102 may be passed to an overhead receiver 115 of the oxygen stripping column 110. In an embodiment, the hydrocarbon feed stream in line 102 may be combined with a cooled overhead stream of the oxygen stripping column in line 114 to provide a combined overhead stream in line 104. The combined overhead stream in line 104 is passed to the overhead receiver 115. From the overhead receiver 115, an overhead liquid stream is taken in line 106 and passed to the oxygen stripping column 110 preferably near the top of the column. Water may be separated in line 107 from the combined overhead stream which is taken from a boot of the overhead receiver 115.

In an embodiment, the overhead liquid stream in line 106 may be heated in a heat exchanger 12 by heat exchange with a purified hydrocarbon feed stream in line 121 to provide a heated reflux stream in line 116 and a cooled purified hydrocarbon feed stream in line 122. The heated reflux stream in line 116 may be recycled to the oxygen stripping column 110. In the oxygen stripping column 110, the heated reflux stream in line 116 comprising the hydrocarbon feed stream in line 102 is stripped of oxygen, oxygenated hydrocarbons and water. An overhead stream containing oxygen, oxygenated hydrocarbons and water is produced in line 112 and cooled in a cooler such as an air cooler 113 to provide the cooled overhead stream in line 114. In the receiver 115, oxygen species are separated from the cooled overhead stream in line 114 and taken in an off-gas stream in line 108.

A stripped hydrocarbon stream is produced from the bottom of the oxygen stripping column 110 in line 111. A reboiling stream is taken from the stripped hydrocarbon stream in line 117 and heated in a reboiler 11 with steam. The steam to the reboiler may be taken from any suitable source. For example, steam may be taken from the process 101 or steam may be taken from any external source. A reboiled stream in line 119 is passed back to the oxygen stripping column 110 near the bottoms. The remainder of the stripped hydrocarbon stream is taken in line 118 and passed to a guard bed 120. In an embodiment, the guard bed 120 may comprise one or both of zinc oxide and copper oxide. In an aspect, the guard bed 120 may include a sulfur guard bed or a chloride treater or both. The purified hydrocarbon feed stream depleted of sulfur is taken in line 121 from the sulfur guard bed 120. The purified hydrocarbon feed stream in line 121 is heat exchanged with the overhead liquid stream in line 116 in the heat exchanger 12 to provide a cooled purified hydrocarbon feed stream in line 122 which is passed to the hydrogenation section 131.

In an exemplary embodiment, the hydrogenation section 131 comprises four hydrogenation reactors, a first hydrogenation reactor 130, a second hydrogenation reactor 140, a third hydrogenation reactor 150, and a fourth hydrogenation reactor 160. In another exemplary embodiment, the hydrogenation section 131 may comprise a polishing reactor which may be the fourth hydrogenation reactor 160. Fewer or more hydrogenation reactors than four may be utilized.

In an aspect, the cooled purified hydrocarbon feed stream in line 122 may fed to a feed manifold 151 which divides the purified hydrocarbon feed stream into a first feed stream in line 123, a second feed stream in line 126, a third feed stream in line 128, and a fourth feed stream in line 129.

The first feed stream in line 123 is charged to a first hydrogenation reactor 130. In an embodiment, a hydrogen stream in line 241, as described later in detail, and a recycle stream in line 175 is combined with the first feed stream in line 123 to provide a combined first feed stream in line 176 which is charged to the first hydrogenation reactor 130. In an embodiment, the combined first feed stream in line 176 is heated in a first effluent heat exchanger 14 by heat exchange with a first hydrogenated effluent stream in line 132 to provide a heated first feed stream in line 124. The heated first feed stream in line 124 may be further heated in a start-up heater 13 to provide a twice heated first feed stream in line 125 which is charged to the first hydrogenation reactor 130. In the first hydrogenation reactor 130, the unsaturated hydrocarbon such as toluene present in the first feed stream is hydrogenated in the presence of hydrogen and a hydrogenation catalyst to produce a first hydrogenated effluent stream comprising a naphthene such as methylcyclohexane. The first hydrogenated effluent stream comprising a naphthene such as methylcyclohexane is discharged in line 132 from the first hydrogenation reactor 130.

Any suitable hydrogenation catalysts may be used in the first hydrogenation reactor 130. The hydrogenation catalyst should have high selectivity and a low rate of coke lay down. Suitable hydrogenation catalysts for the first hydrogenation reactor 130 may include, but are not limited to, a metal of Group VIII of the Periodic Table and optionally a metal of Group I of the Periodic Table. Suitable hydrogenation catalysts for the first hydrogenation reactor 130 may also include, but are not limited to, 0.05 wt % to 30 wt % of a metal of Group VIII of the Periodic Table and optionally 0.1 wt % to 3 wt % of a metal of Group I of the Periodic Table.

The first hydrogenation reactor 130 may be operated at a pressure of about 1034 kPag (150 psig) to about 6895 kPag (1000 psig), or about 2068 kPag (300 psig) to about 4137 (600 psig). The first hydrogenation reactor 130 may be operated at an inlet temperature of about 150° C. (302° F.) to about 232° C. (450° F.). The first hydrogenation reactor 130 may be operated at an outlet temperature of about 270° C. (518° F.) to about 371° C. (700° F.). During turndown, the first hydrogenation reactor 130 may be operated at an outlet temperature of about 204° C. (400° F.) to about 371° C. (700° F.).

The hydrogenation of the unsaturated hydrocarbon such as toluene in the first hydrogenation reactor 130 in the presence of the hydrogenation catalysts is performed at a relatively high reaction temperature. The first hydrogenated effluent stream in line 132 exits the first hydrogenation reactor 130 at an elevated temperature. Heat can be recovered from the first hydrogenated effluent stream in line 132 which may be utilized in the process 101 or exported to other locations. In an aspect, the first hydrogenated effluent stream in line 132 may be passed to the first effluent heat exchanger 14 to heat up the combined first feed stream in line 176 by heat exchange. A cooled first hydrogenated effluent stream in line 133 is discharged from the first effluent heat exchanger 14. In an alternate embodiment, the first hydrogenated effluent stream in line 132 may be passed to a first effluent steam generator 14 to cool the first hydrogenated effluent stream and produce steam. In the first effluent steam generator 14, the first hydrogenated effluent stream in line 132 is indirectly heat exchanged with a boiler feed water stream to convert the boiler feed water stream into steam. In this alternate embodiment, the combined first feed stream in line 176 may be passed directly to the start-up heater 13 and heated therein.

The cooled first hydrogenated effluent stream in line 133 may be further heat exchanged with a once cooled second hydrogenated effluent stream in line 144 in a second effluent heat exchanger 15 to provide a heated first hydrogenated effluent stream in line 134 which is passed to the second hydrogenation reactor 140 and a twice cooled second hydrogenated effluent stream in line 145.

In an embodiment, the heated first hydrogenated effluent stream in line 134 is combined with the second feed stream in line 126 to provide a combined second feed stream in line 136 which is charged to the second hydrogenation reactor 140. In the second hydrogenation reactor 140, the unsaturated hydrocarbon such as toluene present in the second feed stream in line 126 and in the first hydrogenated effluent stream in line 134 is hydrogenated in the presence of hydrogen and a hydrogenation catalyst to produce a second hydrogenated effluent stream comprising a saturated hydrocarbon, perhaps a naphthene such as methylcyclohexane. Hydrogen for the second hydrogenation reaction in the second hydrogenation reactor 140 may be present in the first hydrogenated effluent stream in line 132. Fresh make-up hydrogen may be supplemented to the second hydrogenation reactor 140, but this is not preferred. The second hydrogenated effluent stream is discharged in line 142 from the second hydrogenation reactor 140.

Any suitable hydrogenation catalysts may be used in the second hydrogenation reactor 140. The second hydrogenation reactor 140 may comprise one or more of the hydrogenation catalyst as previously described. The second hydrogenation reactor 140 may comprise a similar or a different hydrogenation catalyst than the first hydrogenation reactor 130.

The second hydrogenation reactor 140 may be operated at a pressure of about 1034 kPag (150 psig) to about 6895 kPag (1000 psig), or about 2068 kPag (300 psig) to about 4137 (600 psig). The second hydrogenation reactor 140 may be operated at an inlet temperature of about 150° C. (302° F.) to about 232° C. (450° F.). The second hydrogenation reactor 140 may be operated at an outlet temperature of about 270° C. (518° F.) to about 371° C. (700° F.). During turndown, the second hydrogenation reactor 140 may be operated at an outlet temperature of about 204° C. (400° F.) to about 371° C. (700° F.). The second hydrogenation reactor 140 may be operated at similar or different operating pressure and temperature than the first hydrogenation reactor 130.

The second hydrogenated effluent stream in line 142 exits the reactor at a high temperature. Heat can be recovered from the second hydrogenated effluent stream in line 142 which may be utilized in the process 101 or exported to other locations.

In accordance with the present disclosure, the second hydrogenated effluent stream in line 142 may be passed to a first steam generator 22 to produce steam. In the first steam generator 22, the second hydrogenated effluent stream in line 142 is indirectly heat exchanged with a first boiler feed water stream in line 194 to convert the first boiler feed water stream into steam. A first steam stream is taken in line 197 from the first steam generator 22. Various levels of steam generation can be accommodated in the first steam generator 22. Low pressure steam is typically generated at about 241 kPag (35 psig) to about 448 kPag (65 psig). Medium pressure steam is typically generated at about 2413 kPag (350 psig) to about 3275 kPag (475 psig) and high pressure steam is typically generated at greater than about 4137 kPag (600 psig). In an exemplary embodiment, the first steam stream in line 197 is a first low pressure steam stream.

In an aspect of the present disclosure, the first steam stream in line 197 is passed to the hydrogen production section 211 to produce hydrogen which is passed to the hydrogenation section 131.

Referring back to the first steam generator 22, a once cooled second hydrogenated effluent stream in line 144 from the first steam generator 22 is charged to the third hydrogenation reactor 150. In an aspect, the once cooled second hydrogenated effluent stream in line 144 may be heat exchanged with the cooled first hydrogenated effluent stream in line 133 in the second effluent heat exchanger 15 to provide the heated first hydrogenated effluent stream in line 134 and the twice cooled second hydrogenated effluent stream in line 145 which is charged to the third hydrogenation reactor 150.

In an embodiment, the twice cooled second hydrogenated effluent stream in line 145 is combined with the third feed stream in line 128 to provide a combined third feed stream in line 146 which is charged to the third hydrogenation reactor 150. In the third hydrogenation reactor 150, the unsaturated hydrocarbon such as toluene present in the third feed stream in line 128 and in the twice cooled second hydrogenated effluent stream in line 145 is hydrogenated in the presence of hydrogen and a hydrogenation catalyst to produce a third hydrogenated effluent stream comprising a saturated hydrocarbon, perhaps a naphthene such as methylcyclohexane. Hydrogen for the third hydrogenation reaction in the third hydrogenation reactor 150 may be present in the second hydrogenated effluent stream in line 142. Fresh make-up hydrogen may be supplemented to the third hydrogenation reactor 150, but this is not preferred. The third hydrogenated effluent stream is discharged in line 152 from the third hydrogenation reactor 150.

Any suitable hydrogenation catalysts may be used the third hydrogenation reactor 150. The third hydrogenation reactor 150 may comprise one or more of the hydrogenation catalysts as previously described. The third hydrogenation reactor 150 may comprise a similar or a different hydrogenation catalyst than the first hydrogenation reactor 130 and/or the second hydrogenation reactor 140.

The third hydrogenation reactor 150 may be operated at a pressure of about 1034 kPag (150 psig) to about 6895 kPag (1000 psig), or about 2068 kPag (300 psig) to about 4137 (600 psig). The third hydrogenation reactor 150 may be operated at an inlet temperature of about 150° C. (302° F.) to about 232° C. (450° F.). The third hydrogenation reactor 150 may be operated at an outlet temperature of about 270° C. (518° F.) to about 371° C. (700° F.). During turndown, the third hydrogenation reactor 150 may be operated at an outlet temperature of about 204° C. (400° F.) to about 371° C. (700° F.). The third hydrogenation reactor 150 may be operated at similar or different operating pressure and temperature than the first hydrogenation reactor 130 and/or the second hydrogenation reactor 140.

The third hydrogenated effluent stream in line 152 exits the reactor at a high temperature. Heat can be recovered from the third hydrogenated effluent stream in line 152 which may be utilized in the process 101 or exported to other locations.

In accordance with the present disclosure, the third hydrogenated effluent stream in line 152 may be passed to a second steam generator 24 to produce steam. In the second steam generator 24, the third hydrogenated effluent stream in line 152 is indirectly heat exchanged with a second boiler feed water stream in line 195 to convert the second boiler feed water stream into steam. A second steam stream is taken in line 198 from the second steam generator 24. Various levels of steam generation can be accommodated in the second steam generator 24. In an exemplary embodiment, the second steam stream in line 198 is a second low pressure steam stream.

In an aspect of the present disclosure, the second steam stream in line 198 is passed to the hydrogen production section 211 to produce hydrogen which is passed to the hydrogenation section 131.

Referring back to the second steam generator 24, a cooled third hydrogenated effluent stream in line 153 from the second steam generator 24 is charged to the fourth hydrogenation reactor 160.

In an embodiment, the cooled third hydrogenated effluent stream in line 153 is combined with the fourth feed stream in line 129 to provide a combined fourth feed stream in line 154 which is charged to the fourth hydrogenation reactor 160. In an exemplary embodiment, the fourth hydrogenation reactor 160 is a polishing reactor.

In the fourth hydrogenation reactor 160, the unsaturated hydrocarbon such as toluene present in the fourth feed stream in line 129 and in the third hydrogenated effluent stream in line 153 is hydrogenated in the presence of hydrogen and a hydrogenation catalyst to produce a fourth hydrogenated effluent stream comprising a saturated hydrocarbon, perhaps a naphthene such as methylcyclohexane. Hydrogen for the fourth hydrogenation reaction in the fourth hydrogenation reactor 160 may be present in the third hydrogenated effluent stream in line 152. Fresh make-up hydrogen may be supplemented to the fourth hydrogenation reactor 160, but this is not preferred. The fourth hydrogenated effluent stream is discharged in line 162 from the fourth hydrogenation reactor 160.

Any suitable hydrogenation catalysts may be used in the fourth hydrogenation reactor 160. The fourth hydrogenation reactor 160 may comprise one or more of the hydrogenation catalyst as previously described. The fourth hydrogenation reactor 160 may comprise a similar or a different hydrogenation catalyst than the first hydrogenation reactor 130 and/or the second hydrogenation reactor 140 and/or the third hydrogenation reactor 150.

The fourth hydrogenation reactor 160 may be operated at a pressure of about 1034 kPag (150 psig) to about 6895 kPag (1000 psig), or about 2068 kPag (300 psig) to about 4137 (600 psig). The fourth hydrogenation reactor 160 may be operated at an inlet temperature of about 150° C. (302° F.) to about 232° C. (450° F.). The fourth hydrogenation reactor 160 may be operated at an outlet temperature of about 270° C. (518° F.) to about 371° C. (700° F.). During turndown, the fourth hydrogenation reactor 160 may be operated at an outlet temperature of about 204° C. (400° F.) to about 371° C. (700° F.). The fourth hydrogenation reactor 160 may be operated at similar or different operating pressure and temperature than the first hydrogenation reactor 130 and/or the second hydrogenation reactor 140 and/or the third hydrogenation reactor 150.

The fourth perhaps polished hydrogenated effluent stream in line 162 exits the fourth hydrogenation reactor 160 at a temperature of about the outlet temperature of the fourth hydrogenation reactor 160. Heat can be recovered from the fourth hydrogenated effluent stream in line 162 which may be utilized in the process 101 or exported to other locations.

In accordance with the present disclosure, the fourth hydrogenated effluent stream in line 162 may be passed to a third steam generator 26 to produce steam. In the third steam generator 26, the fourth hydrogenated effluent stream in line 162 is indirectly heat exchanged with a third boiler feed water stream in line 196 to convert the third boiler feed water stream into steam. A third steam stream is taken in line 199 from the third steam generator 26. In an exemplary embodiment, the third steam stream in line 199 is a third low pressure steam stream.

In an aspect of the present disclosure, the third steam stream in line 199 is passed to the hydrogen production section 211 to produce hydrogen which is charged to the hydrogenation section 131.

In accordance with the present disclosure, the first steam stream in line 197, the second steam stream in line 198, and the third steam stream in line 199 may be combined to provide a combined steam stream in line 202. The combined steam stream may be at low pressure. The combined steam stream in line 202 may be split, for example chemically, in the hydrogen production section 211 to produce hydrogen. In an aspect, the steam for the reboiler 11 may be taken from the combined steam stream in line 202.

The hydrogen production section 211, may comprise an electrolyzer 210, one or more heat exchangers and/or coolers and/or heaters, one or more compressors, and a pressure swing adsorption unit 230. In an embodiment, the combined low pressure steam stream in line 202 may be separated into a first combined low pressure steam stream in line 203 and a second combined low pressure steam stream in line 204. In an embodiment, more than 50% of the combined low pressure steam stream in line 202 is taken in the first combined low pressure steam stream in line 203. In an exemplary embodiment, the about 60% to about 90% of the combined low pressure steam stream in line 202 is taken in the first combined low pressure steam stream in line 203 and about 10% to about 40% of the combined low pressure steam stream in line 202 is taken in the second combined low pressure steam stream in line 204.

The first combined low pressure steam stream in line 203 is heat exchanged with a product hydrogen stream in line 212 in a first heat exchanger 31 to heat it and provide a heated first combined steam stream in line 205 and a cooled product hydrogen stream in line 214. The second combined low pressure steam stream in line 204 is heat exchanged with a product oxygen stream in line 213 in a second heat exchanger 32 to heat it and provide a heated second combined steam stream in line 206 and a cooled product oxygen stream in line 215. The heated first combined steam stream in line 205 and the heated second combined steam stream in line 206 are combined to provide a heated combined steam stream in line 208. The heated combined steam stream in line 208 is passed to the electrolyzer 210.

In an aspect, the electrolyzer 210 may be a high temperature, low pressure electrolyzer. In an exemplary embodiment, the high temperature, low pressure electrolyzer 210 is a solid oxide electrolyzer. The solid oxide electrolyzer 210 may have an electrolyte-supported, electrode-supported, or metal-supported configuration. In another exemplary embodiment, the electrolyzer 210 may be selected from alkaline electrolyzer, proton exchange membrane (PEM), and anion exchange membrane (AEM). The alkaline electrolyzer, PEM, and AEM electrolyzers operate at low temperatures, typically about 80° C. to about 100° C. Solid oxide electrolyzers typically require higher temperatures, but solid oxide electrolyzers have a higher efficiency. Since the LOHC hydrogenation section 131 produces significant amount of heat, solid oxide electrolyzers have an advantage over the other types of electrolyzers in this context.

The solid oxide electrolyzer includes an anode, a cathode and may comprise a solid ceramic (oxide) electrolyte. Solid oxide electrolyzers may include an electrolyzer that produces hydrogen by using electrical energy to break apart the molecular bonds and drive apart the elemental ions that separate into outlet product streams. Solid oxide electrolyzers may have a porous cathode with a porous electrolyte that is catalytic when operated at temperatures at or above 800° C. Catalysis and thermal energy both contribute to cracking the molecular bonds.

In an embodiment, the solid oxide electrolyzer 210 may be operated at a temperature of about 400° C. (752° F.) to about 1000° C. (1832° F.) or about 700° C. (1292° F.) to about 900° C. (1652° F.) or about 400° C. (1112° F.) to about 700° C. (1292° F.) and a pressure of about 100 kPa (g) (14.5 psig) to about 3000 kPa (g) (435 psig) or about 100 kPa (g) (14.5 psig) to about 1000 kPa (g) (145 psig) or about 100 kPa (g) (14.5 psig) to about 300 kPa (g) (43.5 psig).

In an exemplary embodiment, the solid oxide electrolyzer 210 may be an oxygen ion (O²⁻) conducting type. The oxygen ion conducting type electrolyzer 210 may be operated at a temperature of about 700° C. (1292° F.) to about 900° C. (1652° F.). However, some perovskite or double perovskite materials can allow lower temperatures. The oxygen ion conducting type electrolyzer 210 may comprise an electrolyte selected from yttria-stabilized zirconia (YSZ), scandia-stabilized zirconia (ScSZ), or other doped zirconium dioxide (ZrO2), or gadolinium-doped ceria (GDC), or other doped ceria. The oxygen ion conducting type electrolyzer 210 may comprise an anode which may be made from lanthanum strontium manganite (LSM) or YSZ composite, or lanthanum strontium cobalt ferrite (LSCF) or strontium- and magnesium-doped lanthanum gallium oxide (LSGM), or other perovskite or double perovskite materials. The oxygen ion conducting electrolyzer 210 may comprise a cathode which may be made from Ni/YSZ or other Ni cermet materials, or lanthanum strontium chromium manganese oxide or other perovskite materials.

In another exemplary embodiment, the solid oxide electrolyzer 210 may be a proton-conducting type. The proton-conducting type electrolyzer 210 may be operated at a temperature of about 400° C. (1112° F.) to about 700° C. (1292° F.). The proton-conducting type electrolyzer 210 may comprise an electrolyte selected from ABO₃ (A is barium or strontium; B is cerium or zirconium) perovskite proton-conducting oxides or other proton-conducting oxides. The proton-conducting type electrolyzer 210 may comprise an anode which may be made from platinum or non-platinum group metal oxides, such as samarium/strontium/cobalt oxide and others. The proton-conducting electrolyzer 210 may comprise a cathode which may be made from platinum or nickel or nickel based oxide composites.

Power 209 from a power source may also be supplied to the electrolyzer 210. In an embodiment, the power 209 may be green power taken from a renewable source. Power used in electrolyzer 210 includes the conversion of green power to thermal energy. In the electrolyzer 210, the heated combined steam stream in line 208 is separated into its constituents hydrogen and oxygen. Hydrogen ions are deposited on the cathode and the oxygen ions are deposited on the anode. The product hydrogen stream is taken in line 212 from the electrolyzer 210. The product hydrogen stream in line 212 may be at a pressure of about the pressure of the solid oxide electrolyzer 210. The product oxygen stream is taken in line 213 from the electrolyzer 210. The product oxygen stream in line 213 is heat exchanged with the second combined low pressure steam stream in line 204 in the second heat exchanger 32 to provide a cooled oxygen stream in line 215 and the heated second combined low pressure steam stream in line 206.

In an aspect, the hydrogen production section 211 may comprise more than one electrolyzer 210 to meet the hydrogen requirement of the process 101.

The product hydrogen stream is taken in line 212 from the electrolyzer 210 and heat exchanged with the first combined low pressure steam stream in line 203 in the first heat exchanger 31 to provide the cooled product hydrogen stream in line 214 and the heated first combined low pressure steam stream in line 205. The cooled product hydrogen stream in line 214 may be combined with a tail gas stream in line 231 to provide a combined product hydrogen stream in line 239. The combined product hydrogen stream in line 239 may be cooled in a cooler 33 to provide a cooled product hydrogen stream in line 216. The cooled product hydrogen stream in line 216 may be passed to a first knock-out drum (KOD) 220 to separate water from the cooled hydrogen stream in line 216. Water is separated and taken in line 225 from the first KOD 220. A dehydrated hydrogen stream is taken in line 222 and passed to a first booster compressor 223 to provide a first compressed hydrogen stream in line 224. In an embodiment, the dehydrated hydrogen stream in line 222 is compressed to a pressure of about 620 kPag (90 psig) to about 897 kPag (130 psig) or about 689 kPag (100 psig) to about 828 kPag (120 psig). The first compressed hydrogen stream in line 224 is further cooled in a cooler 34 to provide a cooled and compressed first hydrogen stream in line 226. The cooled and compressed first hydrogen stream in line 226 is passed to a second knock-out drum KOD 260 to separate water from the cooled and compressed first hydrogen stream in line 226. Water is separated and taken in line 262 from the second KOD 260. A dehydrated first hydrogen stream is taken in line 261 and compressed in a second booster compressor 227 to provide a second compressed hydrogen stream in line 228. In an embodiment, the dehydrated first hydrogen stream in line 261 is compressed to a pressure of about 1310 kPag (190 psig) to about 1724 kPag (250 psig) or about 1378 kPag (200 psig) to about 1586 kPag (230 psig).

In an exemplary embodiment, the second compressed hydrogen stream in line 228 may be cooled in a cooler 189 to provide a cooled second compressed hydrogen stream in line 229. The cooled second compressed hydrogen stream in line 229 is passed to a third knock-out drum KOD 263 to separate water from the cooled second compressed hydrogen stream in line 229. Water is separated and taken in line 265 from the third KOD 263. A dehydrated second hydrogen stream is taken in line 264 from the third KOD 263. The dehydrated second hydrogen stream in line 264 is processed in the PSA unit 230 to purify the hydrogen by removing any impurities present in the dehydrated second hydrogen stream in line 264 to provide a purified or a hydrogen rich stream in line 232.

The PSA unit 230 includes a series of multiple adsorbent beds containing one or a combination of multiple adsorbents suitable for adsorbing the particular components to be adsorbed therein. These adsorbents include, but are not limited to, activated alumina, silica gel, activated carbon, zeolite molecular sieve type materials, or any combination thereof. The adsorbents are organized in any sequence as required by the adsorption process to adsorb impurities. In an embodiment, the PSA unit 230 includes a molecular sieve for removing water and a catalyst for converting oxygen to water. In the PSA unit 230, the compressed hydrogen stream in line 228 flows over the adsorbents and the larger impurities are adsorbed during the adsorption step while hydrogen passes through in line 232. Periodically, flow to the adsorbent bed is terminated, pressure reduced, so the impurities will exit the bed in a tail gas stream in line 231.

The tail gas stream in line 231 may be recycled. In an embodiment, the tail gas stream in line 231 in combined with the cooled product hydrogen stream in line 214 and recycled as previously described. The hydrogen rich stream in line 232 is charged to the hydrogenation section 131. The hydrogen rich stream in line 232 may be compressed before passing it to the hydrogenation section 131.

In an alternate embodiment, the dehydrated second hydrogen stream in line 264 may be processed in a temperature swing adsorption (TSA) unit 230 to purify the hydrogen and provide a purified or a hydrogen rich stream in line 232.

In an embodiment, the hydrogen rich stream in line 232 may be compressed in a make-up gas compressor 233 to provide a first compressed hydrogen rich stream in line 236. The hydrogen rich stream in line 232 is combined with a hydrogen rich permeate stream in line 172 as described later in detail, to provide a first combined hydrogen rich stream in line 234. The first combined hydrogen rich stream in line 234 is compressed in the make-up gas compressor 233 to provide a first compressed hydrogen rich stream in line 236. The first compressed hydrogen rich stream in line 236 is combined with a second overhead vapor stream in line 179 to provide a second combined hydrogen rich stream in line 237. The second combined hydrogen rich stream in line 237 is compressed in a recycle gas compressor 238 to provide a second compressed hydrogen rich stream in line 241 which is passed to the hydrogenation section 131. The second combined hydrogen rich stream in line 237 may be compressed in the recycle gas compressor 238 to a pressure of about 2413 kPag (350 psig) to about 3793 kPag (550 psig) or about 2068 kPag (300 psig) to about 3448 kPag (500 psig).

In an exemplary embodiment, the twice compressed hydrogen rich stream in line 241 may be combined with the first feed stream in line 123 and passed to the hydrogenation section 131 in the combined first feed stream in line 176.

Referring back to the hydrogenation section 131, a once cooled fourth hydrogenation reactor effluent stream is taken in line 163 from the third steam generator 26. The once cooled fourth hydrogenation reactor effluent stream in line 163 comprises a saturated hydrocarbon, perhaps a naphthene such as methylcyclohexane, which is separated into a product stream. In an embodiment, the once cooled fourth hydrogenation reactor effluent stream in line 163 is separated in a high-pressure separator 170 into a liquid stream comprising a saturated hydrocarbon, perhaps a naphthene such as methylcyclohexane, and a vapor stream comprising hydrogen. In an aspect, the once cooled fourth hydrogenation reactor effluent stream in line 163 is cooled by heat exchange with a cold liquid stream in line 173 in a separator effluent heat exchanger 17 to provide a twice cooled fourth hydrogenation reactor effluent stream in line 164. In another aspect, the twice cooled fourth hydrogenation reactor effluent stream in line 164 is heat exchanged with a boiler feed water stream in line 192 in a feed water preheater 16 to provide a preheated boiler feed water stream in line 193. The preheated boiler feed water stream in line 193 is passed to the steam generators of the hydrogenation section 131. The preheated boiler feed water stream in line 193 is separated into the first boiler feed water stream in line 194, the second boiler feed water stream in line 195, and the third boiler feed water stream in line 196. The first boiler feed water stream in line 194 is passed to the first steam generator 22, the second boiler feed water stream in line 195 is passed to the second steam generator 24, and the third boiler feed water stream in line 196 is passed to the third steam generator 26.

From the feed water preheater 16, a third cooled fourth hydrogenation reactor effluent stream is discharged in line 165. The third cooled fourth hydrogenation reactor effluent stream in line 165 is further cooled in a cooler 19 to provide a fourth cooled fourth hydrogenation reactor effluent stream in line 166. In an embodiment, the cooler 19 is an air-cooled exchanger. In another embodiment, the cooler 19 is a shell and tube heat exchanger. The fourth cooled fourth hydrogenation reactor effluent stream in line 166 is passed to the high-pressure separator 170. An overhead vapor stream comprising hydrogen is taken in line 171 from the high-pressure separator 170. The overhead vapor stream in line 171 may be split into a first overhead vapor stream in line 177 and the second overhead vapor stream in line 179. The first overhead vapor stream in line 177 is passed to a membrane separator 167. A compressed overhead stream in line 188 is also passed to the membrane separator 167. In an embodiment, the first overhead vapor stream in line 177 may be combined with the compressed overhead stream in line 188 and passed to the membrane separator 167 in a combined line. The membrane separator 167 may comprise one or more membranes. In the membrane separator 167, the first overhead vapor stream in line 177 and the compressed overhead stream in line 188 are separated into a hydrogen rich permeate stream in line 172 and a methane rich retentate stream in line 169. The retentate stream in line 169 may be sent to a vent header or a fuel gas header. The hydrogen rich permeate stream in line 172 is combined with the hydrogen rich stream in line 232 to provide the first combined hydrogen rich stream in line 234 which is compressed in the make-up gas compressor 233 to provide the first compressed hydrogen rich stream in line 236. The second overhead vapor stream in line 179 is combined with the first compressed hydrogen rich stream in line 236 to provide the second combined hydrogen rich stream in line 237 and passed to the hydrogenation section 131 as previously described.

A cold liquid stream comprising a saturated hydrocarbon, perhaps a naphthene such as methylcyclohexane is taken from the bottoms of the high-pressure separator 170 in line 173. The cold liquid stream in line 173 passed to the separator effluent heat exchanger 17 to cool the once cooled fourth hydrogenation reactor effluent stream in line 163. In an aspect, the cold liquid stream in line 173 may be separated into a first reheated liquid stream in line 174 and a second reheated liquid stream in line 175. The second reheated liquid stream in line 175 is recycled to the hydrogenation section 131, particularly to the first hydrogenation reactor 130. The second reheated liquid stream in line 175 may be combined with the first feed stream in the first feed line 123 and the hydrogen stream in line 241 and passed to the first hydrogenation reactor 130 in the combined first feed line 176. The second reheated liquid stream in line 175 is combined with the first feed stream in line 123 to dilute the feed before passing the first feed stream in line 123 to the first hydrogenation reactor 140. Diluting the feed with the second reheated liquid stream in line 175 helps in absorbing the exotherm in the hydrogenation section 131 without excessive temperature increase. The first reheated liquid stream in line 174 is further processed to separate the hydrogenated hydrocarbon which may be a naphthene such as methylcyclohexane.

The first reheated liquid stream in line 174 is passed to a stabilizer column 180 to remove dissolved gases that may be present in the first cold liquid stream. The dissolved gases are separated in an overhead stream taken in line 182 from the stabilizer column 180. The stabilizer overhead stream in line 182 is passed to an overhead receiver 250. An off-gas stream comprising the dissolved gases is taken in line 258 from the overhead receiver 250. A condensed liquid stream is taken in line 252 from the overhead receiver 250. A reflux stream is taken in line 256 and recycled back to the top of the stabilizer column 180. The entirety of the condensed liquid stream in line 252 may be refluxed to the stabilizer column 180 in the reflux line 256. Optionally, an overhead liquid stream may be taken in line 254 from the condensed liquid stream. In an embodiment, the overhead liquid stream in line 254 may comprise C₅-C₆ hydrocarbons. The off-gas stream in line 258 is compressed in an off-gas compressor 187 to provide a compressed overhead stream in line 188. The compressed overhead stream in line 188 is passed to the membrane separator 167.

A bottoms stream comprising a hydrogenated hydrocarbon which may be a naphthene such as methylcyclohexane is taken in line 183 from the stabilizer column 180. A reboiling stream is taken in line 185 and reboiled in a reboiler 18 to provide a reboiled stream in line 186 which is recycled back to the bottoms of the stabilizer column 180. A steam stream may be passed to the reboiler 18 to reboil the reboiling stream in line 185. The steam to the reboiler 18 may be taken from any suitable source for example steam may be taken from the process 101 or steam may be taken from any external source. In an aspect, the steam for the reboiler 18 may be taken from the combined low pressure steam stream in line 202.

A hydrogenated product stream comprising saturated hydrocarbon, perhaps naphthene such as methylcyclohexane is taken in a product line 184 from the bottoms of the stabilizer column 180. In an aspect, the product stream in line 184 may be stored and transported to a downstream or a second location for dehydrogenation to unsaturated hydrocarbon such as toluene and hydrogen.

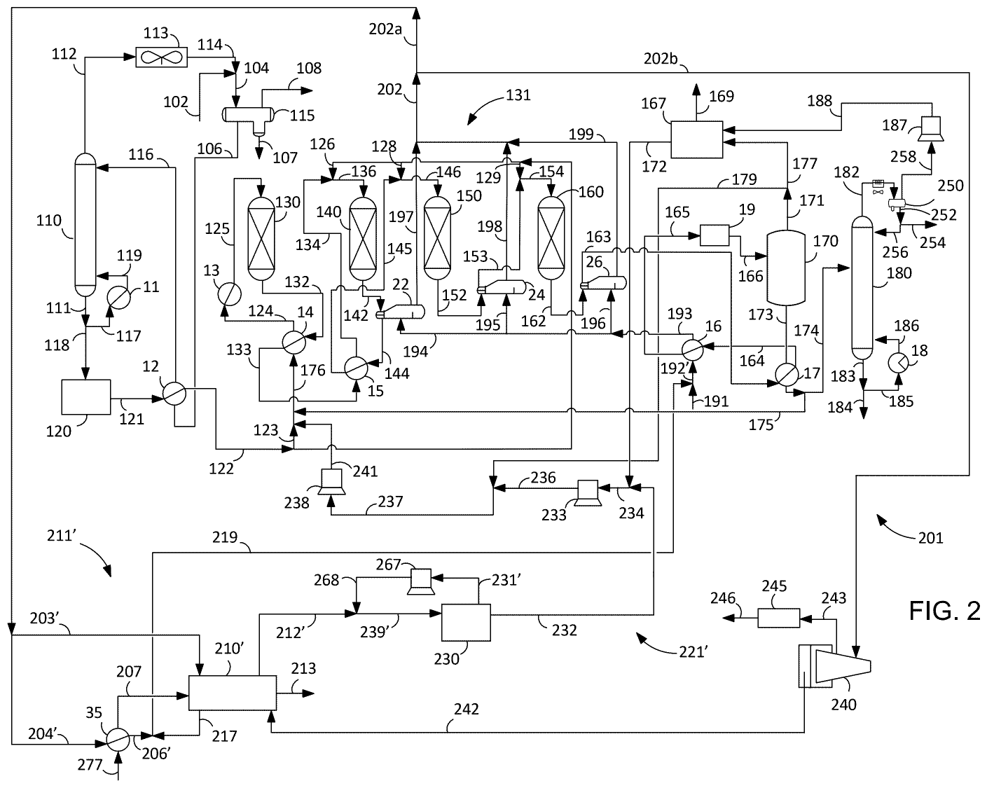

Another embodiment of the process of hydrogenating an unsaturated hydrocarbon 201 is shown in FIG. 2. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

FIG. 2 shows an alternative embodiment to the embodiment of FIG. 1 which employs a turbine to generate power from a portion of the steam stream 202 in line 202b. Also, a portion of the steam stream 202 in line 202a is passed to the electrolyzer 210′ to heat the feed water stream in the electrolyzer 210′.

An alternate embodiment of the process of hydrogenating an unsaturated hydrocarbon such as toluene 201 is shown in FIG. 2. As shown in FIG. 2, the combined steam stream in line 202 may be separated into a first combined steam stream in line 202a and a second combined steam stream in line 202b. The second combined low pressure steam stream in line 202b may be used to produce power particularly green power. In an embodiment, the second combined low pressure steam stream in line 202b is passed to a turbine 240 which may be a steam turbine generator to generate electrical power. Electrical power can be taken from the turbine in electrical line 242 and passed to the electrolyzer 210′ to provide a portion of power to the electrolyzer. An expanded combined steam stream is taken in line 243 from the turbine 240 and cooled in a cooler 245. A third condensate stream may be taken in line 246 from the cooler 245. In an aspect, the third condensate stream in line 246 may be recycled to the hydrogenation section 131 in line 192.

In the embodiment as shown in FIG. 2, the electrolyzer 210′ of the hydrogen production section 211′ may be a low temperature, high pressure electrolyzer. In an exemplary embodiment, the low temperature, high pressure electrolyzer 210′ may be selected from proton exchange membrane (PEM), and anion exchange membrane (AEM). The low temperature, high pressure electrolyzer 210′ may be operated at a temperature of about 20° C. (68° F.) to about 100° C. (212° F.) or about 50° C. (122° F.) to about 80° C. (176° F.) and a pressure of about 100 kPa (g) (14.5 psig) to about 8000 kPa (g) (1160 psig) or about 100 kPa (g) (14.5 psig) to about 3000 kPa (g) (435 psig).

The first combined low pressure steam stream in line 202a is passed to the electrolyzer 210′ to heat the feed water stream to the low temperature, high pressure electrolyzer 210′. The first combined low pressure steam stream in line 202a may be separated into a third combined low pressure steam stream in line 203′ and a fourth combined low pressure steam stream in line 204′. A water feed stream in line 277 is passed to the electrolyzer 210′. The water feed stream in line 277 may be preheated in a feed heater 35. As shown, the fourth combined low pressure steam stream in line 204′ is passed to the feed heater 35 to heat exchange with the water feed stream in line 277 and provide a preheated water feed stream in line 207 and a first condensate stream in line 206′. The first condensate stream in line 206′ may be recycled to the hydrogenation section 131 in line 219. The preheated water feed stream in line 207 is passed to the electrolyzer 210′ and separated into its constituents, hydrogen and oxygen. In the embodiment as shown in FIG. 2, the electrolyzer 210′ may be selected from alkaline electrolyzer, proton exchange membrane (PEM), and anion exchange membrane (AEM). These electrolyzers typically run with liquid water and may not require as much heat as the solid oxide electrolyzer to achieve electrolyzer temperature.

In the electrolyzer 210′, hydrogen ions are deposited on the cathode and the oxygen ions are deposited on the anode. In an embodiment, the third combined low pressure steam stream in line 203′ is passed to the electrolyzer 210′ to further heat the preheated water feed stream in the electrolyzer 210′. A second condensate stream is taken from the electrolyzer 210′ in line 217. The second condensate stream in line 217 is combined with the first condensate stream in line 206′ to provide a combined condensate stream in line 219. In an aspect, the combined condensate stream in line 219 may be recycled back to the hydrogenation section 131. In an embodiment, the water feed stream in line 277 may be taken from the combined condensate stream in line 219. In another embodiment, the combined condensate stream in line 219 may be combined with a boiler feed water stream in line 191 to provide a combined boiler feed water stream in line 192′. The combined boiler feed water stream in line 192′ is passed to the feed water preheater 16 to provide the preheated boiler feed water stream in line 193 which is passed to the hydrogenation section 131 as previously described in FIG. 1.

Referring back to the electrolyzer 210′, the product hydrogen stream is taken in line 212′ from the electrolyzer 210′. The product oxygen stream is taken in line 213 from the electrolyzer 210′. In an aspect, the product hydrogen stream in line 212′ may be at a pressure of about the pressure of the low temperature, high pressure electrolyzer 210. The product hydrogen stream in line 212′ is processed in the PSA unit 230 of the purification section 221′ to purify the hydrogen to provide the hydrogen rich stream in line 232. In the embodiment as shown in FIG. 2, the product hydrogen stream in line 212′ may be combined with a compressed tail gas stream in line 268 to provide a provide a combined product hydrogen stream in line 239′. The combined product hydrogen stream in line 239′ is processed in the PSA unit 230 to produce the hydrogen rich stream in line 232 and the tail gas stream in line 231′. In the embodiment as shown in FIG. 2, the tail gas stream in line 231′ may be compressed in a tail gas compressor 267 to produce a tail gas compressor at a pressure of about the pressure of the low temperature, high pressure electrolyzer 210′. In an exemplary embodiment, the tail gas stream in line 231′ may be compressed to a pressure of about 620 kPag (90 psig) to about 897 kPag (130 psig) or about 689 kPag (100 psig) to about 828 kPag (120 psig) in the tail gas compressor 267. The compressed tail gas stream in line 268 is combined with the product hydrogen stream in line 212′. The hydrogen rich stream in line 232 is passed to the hydrogenation section 131 as previously described in FIG. 1. The rest of the process is same as described in FIG. 1.

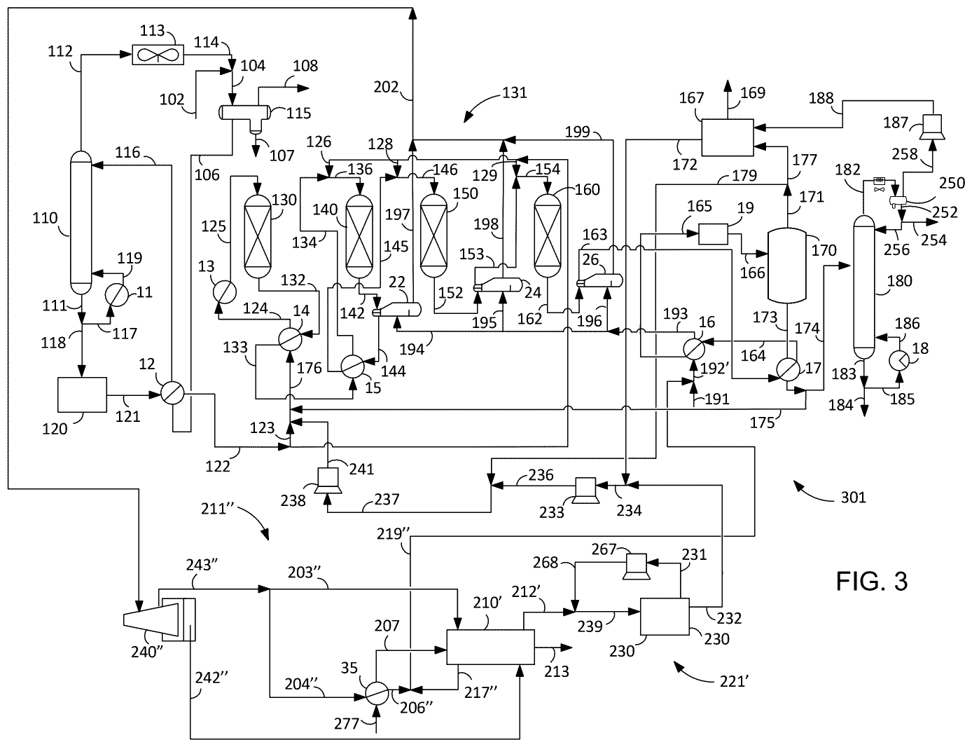

Another embodiment of the process of hydrogenating an unsaturated hydrocarbon 301 is shown in FIG. 3. Elements in FIG. 3 with the same configuration as in FIG. 2 will have the same reference numeral as in FIG. 2. Elements in FIG. 3 which have a different configuration as the corresponding element in FIG. 2 will have the same reference numeral but designated with a double prime symbol (″). The configuration and operation of the embodiment of FIG. 3 is essentially the same as in FIG. 2 with the following exceptions. FIG. 3 shows an alternative embodiment to the embodiment of FIG. 2 which includes an expanded steam stream to generate power from a turbine which is passed to the electrolyzer 210′ of the hydrogen production section 211″.

As shown in FIG. 3, the combined low pressure steam stream in line 202 is passed directly to the turbine 240 to generate electrical power. An expanded combined steam stream is taken in line 243 from the turbine 240 and the electrical power is taken in an electrical line 242″ from the turbine 240. The electrical power in line 242″ is passed to the electrolyzer 210′. The expanded combined steam stream in line 243 is passed to the electrolyzer 210′. The expanded combined steam stream in line 243 is separated into a first expanded steam stream in line 203″ and a second expanded steam stream in line 204″. The second expanded steam stream in line 204″ is passed to the feed heater 35 to heat exchange with the water feed stream in line 277 and provide a preheated water feed stream in line 207 and a cooled second expanded steam stream in line 206″. The preheated water feed stream in line 207 is passed to the electrolyzer 210′ and separated into its constituents hydrogen and oxygen. In an embodiment, the first expanded steam stream in line 203″ is passed to the electrolyzer 210′ to further heat the preheated water feed stream in the electrolyzer 210′. A cooled first expanded steam stream is taken from the electrolyzer 210′ in line 217″. The cooled first expanded steam stream in line 217″ is combined with the cooled second expanded steam stream in line 206″ to provide a combined cooled expanded steam stream in line 219″. The combined cooled expanded steam stream in line 219″ is recycled back to the hydrogenation section 131 as described in FIG. 2. The rest of the process is same as described in FIG. 2.

### Example

A comparative study was performed to compare an independent process with the present process. The present process includes integrating the hydrogenation reactor with the electrolyzer and the independent process included a non-integrated hydrogenation reactor. In the current integrated process, the steam stream was taken from the hydrogenation reactor and passed to the electrolyzer for the thermal energy requirement. Various parameters and the results of the study are listed in Table below.

TABLE

| | |
|---|---|
| Example Hydrogen Capacity: | 100,000 tonne/year |
| On-stream efficiency: | 8000 h/year |
| Hydrogen product rate: | 12500 kg/h |

Electrolysis unit

| | | |
|---|---|---|
| Energy required | Independent process | Integrated process |
| Thermal energy (MW) | 71.1 | 2.0 |
| Electricity (MW) | 424.8 | 424.8 |

Hydrogenation Unit

| | | |
|---|---|---|
| Energy required | Independent process | Integrated process |
| Electricity (MW) | −19.1 | 0 |
| Total energy required = Electrolysis | 476.8 | 426.8 |
| unit + Hydrogenation Unit | | |

As shown in the Table, the independent process with the green energy production facility required about 71.1 MW of thermal energy in the electrolyzer as compared to the only 2.0 MW of thermal energy required in the electrolyzer for the integrated process. For total energy, the integrated process required about 426.8 MW of energy as compared to 476.8 MW required in the independent process with a green energy production facility. Thus, the current integrated process saved energy and is 10.5% more energy efficient than the independent process with the green energy production facility.

### Specific Embodiments

While the following is described in conjunction with specific embodiments, it will be understood that this description is intended to illustrate and not limit the scope of the preceding description and the appended claims.

A first embodiment of the present disclosure is a process of hydrogenating an unsaturated hydrocarbon, comprising passing a hydrocarbon feed stream to a hydrogenation reactor; passing a hydrogen stream to the hydrogenation reactor; hydrogenating the hydrocarbon feed stream in the hydrogenation reactor in the presence of hydrogen and a hydrogenation catalyst to produce a hydrogenated effluent stream; generating a steam stream from the hydrogenation reactor; producing hydrogen from a water stream in an electrolyzer to produce the hydrogen stream and providing heat requirements for the electrolyzer from the steam stream. An embodiment of the present disclosure is one, any or all of prior embodiments in this paragraph up through the first embodiment in this paragraph, wherein the electrolyzer is a solid oxide electrolyzer. An embodiment of the present disclosure is one, any or all of prior embodiments in this paragraph up through the first embodiment in this paragraph further comprising passing the hydrogenated effluent stream to a steam generator to convert a water stream into the steam stream and provide a cooled hydrogenated effluent stream. An embodiment of the present disclosure is one, any or all of prior embodiments in this paragraph up through the first embodiment in this paragraph, wherein the step of hydrogenating the hydrocarbon feed stream in the hydrogenation reactor comprises taking a first feed stream from the hydrocarbon feed stream; passing the first feed stream and the hydrogen stream to a first hydrogenation reactor to hydrogenate toluene in the presence of hydrogen and a first hydrogenation catalyst to produce a first hydrogenated effluent stream; and passing the first hydrogenated effluent stream and a second feed stream taken from the hydrocarbon feed stream to a second hydrogenation reactor to hydrogenate toluene in the presence of hydrogen and a second hydrogenation catalyst to produce a second hydrogenated effluent stream. An embodiment of the present disclosure is one, any or all of prior embodiments in this paragraph up through the first embodiment in this paragraph further comprising passing the second hydrogenated effluent stream and a third feed stream taken from the hydrocarbon feed stream to a third hydrogenation reactor to hydrogenate toluene in the presence of hydrogen and a third hydrogenation catalyst to produce a third hydrogenated effluent stream; and passing the third hydrogenated effluent stream and a fourth feed stream taken from the hydrocarbon feed stream to a fourth hydrogenation reactor of the hydrogenation reactor to produce a fourth hydrogenated effluent stream comprising methylcyclohexane. An embodiment of the present disclosure is one, any or all of prior embodiments in this paragraph up through the first embodiment in this paragraph further comprising separating methylcyclohexane from the fourth hydrogenated effluent stream. An embodiment of the present disclosure is one, any or all of prior embodiments in this paragraph up through the first embodiment in this paragraph further comprising passing the second hydrogenated effluent stream to a first steam generator to convert a first water stream into a first steam stream and provide a cooled second hydrogenated effluent stream; passing the cooled second hydrogenated effluent stream to the third hydrogenation reactor; passing the third hydrogenated effluent stream to a second steam generator to convert a second water stream into a second steam stream and provide a cooled third hydrogenated effluent stream; passing the cooled second hydrogenated effluent stream to the fourth hydrogenation reactor; passing the fourth hydrogenated effluent stream to a third steam generator to convert a third water stream into a third steam stream and provide a cooled fourth hydrogenated effluent stream; and separating methylcyclohexane from the cooled fourth hydrogenated effluent stream. An embodiment of the present disclosure is one, any or all of prior embodiments in this paragraph up through the first embodiment in this paragraph further comprising combining the first steam stream, the second steam stream, and the third steam stream to provide a combined steam stream; passing the combined steam stream to the electrolyzer; and producing hydrogen from the combined steam stream in the electrolyzer to produce the hydrogen stream. An embodiment of the present disclosure is one, any or all of prior embodiments in this paragraph up through the first embodiment in this paragraph further comprising separating the steam stream into a first combined steam stream and a second combined steam stream; heating the first combined steam stream with the hydrogen stream in a heat exchanger to provide a heated first combined steam stream; heating the second combined steam stream with an oxygen stream in a heat exchanger to provide a heated second combined steam stream; and passing the heated first combined steam stream and the heated second combined steam stream to the electrolyzer to produce the hydrogen stream and the oxygen stream. An embodiment of the present disclosure is one, any or all of prior embodiments in this paragraph up through the first embodiment in this paragraph wherein more than 50% of the steam stream is taken in the first steam stream. An embodiment of the present disclosure is one, any or all of prior embodiments in this paragraph up through the first embodiment in this paragraph further comprising passing the cooled fourth hydrogenated effluent stream to a separator to provide a vapor effluent stream and a liquid effluent stream; recycling the vapor effluent stream to the first hydrogenation reactor; and stabilizing the liquid effluent stream in a stabilizer column to produce a product stream comprising methylcyclohexane. An embodiment of the present disclosure is one, any or all of prior embodiments in this paragraph up through the first embodiment in this paragraph further comprising taking a recycle liquid stream from the liquid effluent stream; combining the recycle liquid stream with the hydrocarbon feed stream to provide a combined feed stream; and passing the combined feed stream to the hydrogenation reactor. An embodiment of the present disclosure is one, any or all of prior embodiments in this paragraph up through the first embodiment in this paragraph, further comprising separating the steam stream into a first steam stream and a second steam stream; feeding the first steam stream to the electrolyzer to provide the heat requirements for the electrolyzer; and expanding the second steam stream to provide electrical power requirement for the electrolyzer.

A second embodiment of the present disclosure is a process of hydrogenating an unsaturated hydrocarbon, comprising passing a hydrocarbon feed stream to a hydrogenation reactor; passing a hydrogen stream to the hydrogenation reactor; hydrogenating the hydrocarbon feed stream in the hydrogenation reactor in the presence of hydrogen and a hydrogenation catalyst to produce a hydrogenated effluent stream; taking a steam stream from the hydrogenation reactor; and producing hydrogen from the steam stream in an electrolyzer to provide the hydrogen stream. An embodiment of the present disclosure is one, any or all of prior embodiments in this paragraph up through the second embodiment in this paragraph further comprising one or more steps of compressing, cooling, and dehydrating the hydrogen stream before passing the hydrogen stream to the hydrogenation reactor. An embodiment of the present disclosure is one, any or all of prior embodiments in this paragraph up through the second embodiment in this paragraph further comprising; separating a vapor effluent stream from the hydrogenated effluent stream; combining the vapor effluent stream with the hydrogen stream to provide a combined vapor effluent stream; and passing the combined vapor effluent stream to the hydrogenation reactor. An embodiment of the present disclosure is one, any or all of prior embodiments in this paragraph up through the second embodiment in this paragraph, wherein the electrolyzer is a solid oxide electrolyzer. An embodiment of the present disclosure is one, any or all of prior embodiments in this paragraph up through the second embodiment in this paragraph further comprising passing the hydrogenated effluent stream to a steam generator to convert a water stream into the steam stream and provide a cooled hydrogenated effluent stream. An embodiment of the present disclosure is one, any or all of prior embodiments in this paragraph up through the second embodiment in this paragraph further comprising providing heat requirements for the electrolyzer from the steam stream.

A third embodiment of the present disclosure is a process of hydrogenating an unsaturated hydrocarbon, comprising passing a hydrocarbon feed stream comprising toluene to a hydrogenation reactor operating in a vapor phase; passing a hydrogen stream to the hydrogenation reactor; hydrogenating the hydrocarbon feed stream in the hydrogenation reactor in the presence of hydrogen and a hydrogenation catalyst to produce a hydrogenated effluent stream comprising methylcyclohexane; taking a steam stream from the hydrogenation reactor; and producing hydrogen from the steam stream in an electrolyzer to provide the hydrogen stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of hydrogenating an unsaturated hydrocarbon, comprising:
passing a hydrocarbon feed stream to a hydrogenation reactor;
passing a hydrogen stream to the hydrogenation reactor;
hydrogenating said hydrocarbon feed stream in the hydrogenation reactor in the presence of hydrogen and a hydrogenation catalyst to produce a hydrogenated effluent stream;
generating a steam stream from the hydrogenation reactor;
producing hydrogen from a water stream in an electrolyzer to produce said hydrogen stream; and
providing heat requirements for said electrolyzer from said steam stream.

2. The process of claim 1, wherein the electrolyzer is a solid oxide electrolyzer.

3. The process of claim 1 further comprising:
passing said hydrogenated effluent stream to a steam generator to convert a water stream into said steam stream and provide a cooled hydrogenated effluent stream.

4. The process of claim 1, wherein the step of hydrogenating said hydrocarbon feed stream in the hydrogenation reactor comprises:
taking a first feed stream from said hydrocarbon feed stream;
passing said first feed stream and said hydrogen stream to a first hydrogenation reactor to hydrogenate toluene in the presence of hydrogen and a first hydrogenation catalyst to produce a first hydrogenated effluent stream; and
passing said first hydrogenated effluent stream and a second feed stream taken from said hydrocarbon feed stream to a second hydrogenation reactor to hydrogenate toluene in the presence of hydrogen and a second hydrogenation catalyst to produce a second hydrogenated effluent stream.

5. The process of claim 4 further comprising:
passing said second hydrogenated effluent stream and a third feed stream taken from said hydrocarbon feed stream to a third hydrogenation reactor to hydrogenate toluene in the presence of hydrogen and a third hydrogenation catalyst to produce a third hydrogenated effluent stream; and
passing said third hydrogenated effluent stream and a fourth feed stream taken from said hydrocarbon feed stream to a fourth hydrogenation reactor of the hydrogenation reactor to produce a fourth hydrogenated effluent stream comprising methylcyclohexane.

6. The process of claim 5 further comprising separating methylcyclohexane from said fourth hydrogenated effluent stream.

7. The process of claim 5 further comprising:
passing said second hydrogenated effluent stream to a first steam generator to convert a first water stream into a first steam stream and provide a cooled second hydrogenated effluent stream;
passing said cooled second hydrogenated effluent stream to the third hydrogenation reactor;
passing said third hydrogenated effluent stream to a second steam generator to convert a second water stream into a second steam stream and provide a cooled third hydrogenated effluent stream;
passing said cooled second hydrogenated effluent stream to the fourth hydrogenation reactor;
passing said fourth hydrogenated effluent stream to a third steam generator to convert a third water stream into a third steam stream and provide a cooled fourth hydrogenated effluent stream; and
separating methylcyclohexane from said cooled fourth hydrogenated effluent stream.

8. The process of claim 7 further comprising:
combining said first steam stream, said second steam stream, and said third steam stream to provide a combined steam stream;
passing said combined steam stream to the electrolyzer; and
producing hydrogen from said combined steam stream in the electrolyzer to produce said hydrogen stream.

9. The process of claim 3 further comprising:
separating said steam stream into a first combined steam stream and a second combined steam stream;
heating said first combined steam stream with said hydrogen stream in a heat exchanger to provide a heated first combined steam stream;
heating said second combined steam stream with an oxygen stream in a heat exchanger to provide a heated second combined steam stream; and
passing said heated first combined steam stream and said heated second combined steam stream to the electrolyzer to produce said hydrogen stream and said oxygen stream.

10. The process of claim 9 wherein more than 50% of the steam stream is taken in said first steam stream.

11. The process of claim 7 further comprising:
passing said cooled fourth hydrogenated effluent stream to a separator to provide a vapor effluent stream and a liquid effluent stream;
recycling said vapor effluent stream to the first hydrogenation reactor; and
stabilizing said liquid effluent stream in a stabilizer column to produce a product stream comprising methylcyclohexane.

12. The process of claim 11 further comprising:
taking a recycle liquid stream from said liquid effluent stream;
combining said recycle liquid stream with said hydrocarbon feed stream to provide a combined feed stream; and
passing said combined feed stream to the hydrogenation reactor.

13. The process of claim 1, further comprising:
separating said steam stream into a first steam stream and a second steam stream;
feeding said first steam stream to the electrolyzer to provide the heat requirements for said electrolyzer; and
expanding said second steam stream to provide electrical power requirement for said electrolyzer.

14. A process of hydrogenating an unsaturated hydrocarbon, comprising:
passing a hydrocarbon feed stream to a hydrogenation reactor;
passing a hydrogen stream to the hydrogenation reactor;
hydrogenating said hydrocarbon feed stream in the hydrogenation reactor in the presence of hydrogen and a hydrogenation catalyst to produce a hydrogenated effluent stream;
taking a steam stream from the hydrogenation reactor; and
producing hydrogen from said steam stream in an electrolyzer to provide said hydrogen stream.

15. The process of claim 14 further comprising one or more steps of compressing, cooling, and dehydrating said hydrogen stream before passing said hydrogen stream to the hydrogenation reactor.

16. The process of claim 14 further comprising;
separating a vapor effluent stream from said hydrogenated effluent stream;
combining said vapor effluent stream with said hydrogen stream to provide a combined vapor effluent stream; and
passing said combined vapor effluent stream to the hydrogenation reactor.

17. The process of claim 14, wherein the electrolyzer is a solid oxide electrolyzer.

18. The process of claim 14 further comprising:
passing said hydrogenated effluent stream to a steam generator to convert a water stream into said steam stream and provide a cooled hydrogenated effluent stream.

19. The process of claim 14 further comprising providing heat requirements for said electrolyzer from said steam stream.

20. A process of hydrogenating an unsaturated hydrocarbon, comprising:
passing a hydrocarbon feed stream comprising toluene to a hydrogenation reactor operating in a vapor phase;
passing a hydrogen stream to the hydrogenation reactor,
hydrogenating said hydrocarbon feed stream in the hydrogenation reactor in the presence of hydrogen and a hydrogenation catalyst to produce a hydrogenated effluent stream comprising methylcyclohexane;
taking a steam stream from the hydrogenation reactor, and
producing hydrogen from said steam stream in an electrolyzer to provide said hydrogen stream.
